(19) United States
(12) Patent Application Publication
(10) Pub. No.: US 2019/0154669 A1
(43) Pub. Date: May 23, 2019

(54) METHODS AND SYSTEMS FOR SPECIES-ON-SPECIES IMMUNOASSAY DETECTION

(71) Applicant: Vector Laboratories, Inc., Burlingame, CA (US)

(72) Inventor: Pamela JAMES, Burlingame, CA (US)

(73) Assignee: Vector Laboratories, Inc., Burlingame, CA (US)

(21) Appl. No.: 16/195,208

(22) Filed: Nov. 19, 2018

Related U.S. Application Data
(60) Provisional application No. 62/588,647, filed on Nov. 20, 2017.

(51) Int. Cl. G01N 33/53; G01N 33/542; G01N 33/566

(52) U.S. Cl. CPC G01N 33/5306; G01N 33/566; G01N 33/542

(57) ABSTRACT

The present disclosure generally relates to methods for detecting one or more analytes in a sample using immunoassays. In some embodiments, the disclosure provides methods that include quenching the free secondary antibody with a fractionated serum composition. In some other aspects, the disclosure provides systems and kits suitable for carrying out the foregoing methods and any embodiments thereof.

### CROSS REFERENCE TO RELATED APPLICATION

This application claims priority to U.S. Provisional Application No. 62/588,647 filed Nov. 20, 2017 which is hereby incorporated by reference in its entirety.

### TECHNICAL FIELD

The present disclosure generally relates to methods for detecting one or more analytes in a sample using immunoassays. In some embodiments, the disclosure provides methods that include quenching the free secondary antibody with a fractionated serum composition. In some other aspects, the disclosure provides systems and kits suitable for carrying out the foregoing methods and any embodiments thereof.

### BACKGROUND

Immunoassays provide a practical way to determine the presence or concentration of one or more analytes in a complex sample, such as a biological sample. Immunoassays can detect a variety of different analytes, ranging from proteins, hormones, and other biologically important compounds to contaminants in food.

Immunoassays depend on the ability of a particular antibody (the primary antibody) to bind selectively to one or more particular analytes to the exclusion of other compounds in the sample. In general, antibodies are generated by animals in response to invasion by the analyte or a compound bearing certain features of the analyte. Thus, the antibody binds selectively and non-covalently to the analyte in any environment in which it encounters the analyte. Thus, once generated, these analyte-specific antibodies provide a useful tool for identifying whether an analyte is present, and in what relative concentration it is present. In order to detect the analyte-bound primary antibody in the sample, one can link the primary antibody to a label that is amenable to detection, such as through fluorescence or other suitable means. One way of linking the primary antibody to such labels is through the use of another antibody or antibody fragment (a secondary antibody or secondary reagent). This secondary antibody or reagent includes a detectable label of some sort and also includes an antibody or antibody fragment that contains a portion that binds non-covalently to the primary antibody, preferably in a way that does not interfere with the ability of the primary antibody to bind to the analyte.

Typical implementations of these principles involve a series of steps. The primary antibody is introduced to the sample and binds to any of the analyte present in the sample. Then, following a wash, the labeled secondary antibody is introduced to the sample and binds to any analyte-bound primary antibody that may be present in the sample. However, this poses certain problems when the primary antibody is derived from the same animal species as the sample, as the labeled secondary antibody invariably exhibits some cross-reactivity to endogenous antibodies in the tissue. Thus, such procedures end up producing false positives, as the labeled secondary antibody labels some endogenous antibodies in addition to the analyte-bound primary antibody.

To avoid obtaining such false positives, one may carry out the labeling of the primary antibody prior to incubation with the sample, for example, by introducing the labeled secondary antibody to the primary antibody before introducing the composition containing both primary antibody and labeled secondary antibody to the sample. To deal with the potential for cross-reactivity, one can introduce a quenching reagent to capture the unbound secondary antibodies and prevent them from binding to endogenous antibodies in the sample. Serum or purified Immunoglobulin G (IgG) can be used to carry out such a quenching. This generally reduces some of the cross-reactivity and the resulting false positives. But these solutions are far from perfect. For example, use of serum as the quenching reagent also tends to interfere with the binding of the primary antibody with the analyte. So, the resulting method ends up under-detecting the analyte at the cost of solving the problem with false positives. The use of purified IgG as the quenching reagent does not appear to suffer from these problems, it nevertheless does not entirely solve the problem of false positives.

Thus, there is a continuing need to develop immunoassay methods for detecting analytes in a complex biological sample that minimize the incidence of false positives while maximizing analyte-antibody binding in the sample.

### SUMMARY

The methods, systems, and kits disclosed herein provide an improved means of carrying out indirect immunoassay detection of an analyte. The methods can be carried out with a smaller number of steps in comparison to certain predecessor methods, and can detect analytes in a complex biological sample with a low incidence of false positives and a high incidence of analyte-antibody binding.

In at least one aspect, the disclosure provides methods for determining the presence of a target compound in a sample. The methods comprise providing (a) a target-binding antibody comprising a region that binds non-covalently to a target compound, (b) a quenching composition comprising quenching compounds, wherein the quenching compounds comprise enriched serum components, and wherein the enriched serum components are at a concentration greater than 4-fold that normally found in serum and (c) a labeling composition comprising one or more labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds; introducing the target-binding antibody into the labeling composition to form an active product composition comprising (a) a labeled antibody complex, which comprises the target-binding antibody and one or more labeling proteins bound non-covalently to the target-binding antibody, and (b) free labeling proteins, which are not bound non-covalently to any target-binding antibodies; introducing the quenching composition to the active product composition to form a quenched active product composition comprising the labeled antibody complex and quenched labeling proteins, wherein each of the quenched labeling proteins comprises one or more quenching compounds bound non-covalently to one or more labeling proteins; and introducing at least a portion of the quenched active product composition to a sample; and following the introduction of at least a portion of the quenched active product composition to the sample, analyzing the sample to determine the presence or absence of the target compound in the sample.

The target compound is selected from the group including a protein, an antibody, an amino acid, a peptide, an oligopeptide, a glycoprotein, an enzyme, an enzyme substrate, a hormone, a lymphokine, a metabolite, an antigen, a hapten, a lectin, avidin, streptavidin, a toxin, a poison, an environmental pollutant, a carbohydrate, a carbohydrate, an oligosaccharide, a polysaccharide, a lipid, a glycolipid, a nucleotide, an oligonucleotide, a nucleic acid, a derivatized nucleic acid (such as a deoxyribo- or ribonucleic acid, or a peptide nucleic acid), a DNA fragment, an RNA fragment, a derivatized DNA or RNA fragment (such as a single- or multi-stranded fragment), a natural or synthetic drug, a receptors, a virus particle, a bacterial particle, a virus component, a biological cell, a cellular component (such as a cellular membrane or an organelle), a natural or synthetic lipid vesicle, and a polymer membrane.

In some examples, the sample is a tissue of a mammal. In some cases, the target binding antibody is of the same species as the tissue of the mammal. In some examples, the sample comprises a solid tissue. In other examples, the tissue is a fluid, such as blood, sputum, cerebral fluid, and the like.

In some examples, the monovalent Fab antibody fragment is derived from a monoclonal antibody or a polyclonal antibody. In some cases, the quenching composition comprises albumin at a concentration of no more than 10 mg/mL. In some examples, the quenching composition comprises one or more naturally occurring subclasses of immunoglobulin G. In some examples, the quenching compounds comprise one or more gamma-globulins, which comprise one or more immunoglobulins of an isotype other than immunoglobulin G. In some cases, the one or more gamma-globulins comprise one or more immunoglobulins selected from the group including immunoglobulin A and immunoglobulin M.

In some examples, the method comprises introducing a blocking composition to the sample before introduction of at least a portion of the quenched active product composition to the sample.

In some examples, the blocking composition is substantially free of mammalian antibodies and mammalian antibody fragments. In some cases, the blocking composition comprises a non-animal blocking agent. In some examples, the blocking composition comprises a plant protein. In some examples, the plant protein is a rice protein.

In some examples, the quenching composition is formed by a process comprising providing a starting composition comprising serum of the same species as the tissue; fractionating the starting composition to form an enriched composition comprising fractionated serum proteins comprising alpha globulins, beta globulins, and gamma globulins; and concentrating the fractionated serum proteins to form the quenching composition, where the quenched active product composition comprises a 5000-fold molar excess of the fractionated serum proteins relative to the labeling protein.

In some cases, the fractionating step comprises precipitation with kosmotropic agents, selective precipitants, or fractionating chromatography.

In some examples, a portion of the monovalent Fab antibody fragment binds non-covalently to at least a portion of the target-binding antibody not within an Fc region of the target-binding antibody.

In some examples, the label is selected from the group including a fluorescent dye, a phosphorescent dye, a tandem dye, a particle, a nanoparticle, an electron transfer agent, biotin, a hapten, an enzyme, and a radioisotope.

In at least another aspect, the disclosure provides a systems, the systems comprising: (a) a target-binding antibody comprising a region that binds non-covalently to a target compound, (b) a quenching composition comprising quenching compounds, wherein the quenching compounds comprise serum components, and wherein the serum components are at a concentration greater than that normally found in serum and (c) a labeling composition comprising labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds.

In at least another aspect, the disclosure provides kits, the kits comprising: (a) a quenching composition comprising quenching compounds, wherein the quenching compounds comprise serum components, and wherein the serum components are at a concentration greater than that normally found in serum, and (c) a labeling composition comprising labeling proteins, wherein each of the labeling proteins comprises a monovalent Fab antibody fragment comprising a region that binds non-covalently to a target-binding antibody, and wherein each of the labeling proteins is linked covalently to one or more labels, and wherein each of the labeling proteins is monovalent and capable of binding non-covalently to at least one of the quenching compounds.

Further aspects and embodiments of the disclosure are set forth in the following drawings, detailed description, and claims.

### BRIEF DESCRIPTION OF DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1A-1C shows the performance of certain embodiments of the methods disclosed herein in removing background signal while maintaining specific signal.

FIG. 2A-2C shows the improvement in specific signal with certain embodiments of the methods disclosed herein as compared to an existing method that is commercially available.

FIG. 3 shows the performance of certain embodiments of the methods disclosed herein in removing background signal while maintaining specific signal.

FIG. 4 shows the performance of comparative methods regarding specific signal to background signal.

FIG. 5 shows the performance of comparative methods regarding specific signal to background signal.

FIG. 6 shows the performance of comparative methods regarding specific signal to background signal.

FIG. 7 shows the performance of certain embodiments of the methods disclosed herein in removing background signal.

FIG. 8 shows the performance of a comparative method background signal.

FIG. 9 shows the performance of a comparative method background signal.

FIG. 10 shows the performance of a comparative method background signal.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “introduce” or “introduction” refers to any disposition of a substance of a mixture of substances with another substance or mixture of substances to form a new mixture of substances. In some instances, the introduction can result in a chemical reaction occurring, such that new covalent interactions are formed between atoms. In other instances, however, the introduction dos not result in a chemical reaction occurring. In some such instances, non-covalent interactions may form between certain substances.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the term “provide” or “providing” is to be given its broadest reasonable interpretation, and does not imply that items are provided in a particular way or manner. For example, a technician who arranges certain items for use in conducting a test or experiment is engaged in the act of providing. Moreover, when providing two or more items, the two or more items need not be part of a common package or kit or from a common source.

As used herein, “antibody” refers to a protein of the immunoglobulin (Ig) superfamily. In general, antibodies bind non-covalently and selectively to certain substances to form a complex. Antibodies can be produced by a variety of means, including but not limited to production by hybridoma cell lines, by immunization of an animal to elicit a polyclonal antibody response, by chemical synthesis, and by recombinant host cells that have been transformed with an expression vector that encodes the antibody. Human immunoglobulin antibodies are classified as IgA, IgD, IgE, IgG, and IgM, and members of each class are said to have the same “isotype.” Human IgA and IgG isotypes are further subdivided into subtypes, for example, IgA₁, and IgA₂, IgG₁, and the like. The term “antibody,” as used herein, includes any of the various classes (isotypes) and sub-classes of immunoglobulin derived from any of the animals conventionally used, as well as polyclonal and monoclonal antibodies, such as murine, chimeric, or humanized antibodies. Antibody molecules have regions of amino acid sequences that can act as an antigenic determinant, for example, the Fc region, the kappa light chain, the lambda light chain, the hinge region, and the like. An antibody is typically generated against an antigen by immunizing an organism with a macromolecule to initiate lymphocyte activation to express the immunoglobulin protein. The term antibody, as used herein, also includes any polypeptide or other protein having a binding domain that is, or is homologous to, an antibody binding domain, such as single-chain Fv molecules (scFv).

As used herein, “antibody fragment” refers to any fragment of an antibody that retains the principal selective binding characteristics of the whole antibody. Non-limiting examples of antibody fragments include Fab, Fab′, and F(ab′)₂ fragments, which are obtained by digestion with various proteases, and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include the Fd fragment, which has the VH and CH1 domains, the dAb fragment, which has a VH domain, isolated CDR regions, and single-chain Fv molecules (scFv). The term antibody fragment also includes any fragments made using recombinant technology, and which retain antigen-recognition characteristics.

As used herein, “antigen” refers to a molecule that induces, or is capable of inducing, the formation of an antibody, or to which an antibody binds selectively. In some instances, the antigen is a material of biological origin, such as compounds found naturally within certain animal species. But the term antigen can also refer to compounds not naturally found within certain animal species, and which may be foreign to the animal species or otherwise synthetic in origin. The term antigen also refers to an “immunogen.” An antibody binds selectively to an antigen when there is a relative lack of cross-reactivity with or interference by other substances present. For example, in some cases there can be less than 3% cross-reactivity with or interference by other substances present. In other cases, there can be less than 2%, less than 1%, less than 0.5%, less than 0.2%, less than 0.1% or less than 0.01% cross-reactivity with or interference by other substances present.

As used herein, “complex” refers to two or more molecules held together by non-covalent bonding, which are typically non-covalent combinations of biomolecules, such as a protein complexed with another protein. By contrast, a protein is covalently labeled with a substance when there is a covalent chemical bond between the substance and the protein.

As used herein, “label” refers to a chemical substance to facilitate identification or quantitation of a target analyte. This includes labels that can be directly observed or measured, as well as those that can be indirectly observed or measured. Such labels include, but are not limited to, the following: radiolabels that can be measured with radiation-counting devices; pigments, dyes or other chromogens that can be visually observed or measured with optical devices, such as a spectrophotometer; spin labels that can be measured with a spin label analyzer; and fluorescent moieties, where the output signal is generated by excitation of a suitable molecular adduct after excitation with light that is absorbed by the dye, where the output signal can be measured with standard fluorometers or imaging systems, for example. The label can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The label may also take the form of a chemical or biochemical, or an inert particle, including but not limited to, colloidal gold, microspheres, quantum dots, or inorganic crystals such as nanocrystals or phosphors. The term label can also refer to a tag or hapten that can bind selectively to a labeled molecule, such that the labeled molecule, when added subsequently, can be used to generate a detectable signal. For instance, one can use biotin, iminobiotin, or desthiobiotin as a tag and then use an avidin or streptavidin conjugate of horseradish peroxidase (HRP) to bind to the tag, and then use a chromogenic substrate (such as, for example, tetramethylbenzidine) or a fluorogenic substrate such as Amplex Red or Amplex Gold (Thermo Fisher, Waltham, Mass.) to detect the presence of HRP. In other examples, the tag can be a hapten or antigen (such as digoxigenin), and an enzymatically, fluorescently, or radioactively labeled antibody can also be used to bind to the tag. Numerous labels are known by those of skill in the art and include, but are not limited to, particles, fluorescent dyes, haptens, enzymes and their chromogenic, fluorogenic, and chemiluminescent substrates, and other labels that are described in Haugland, Richard P. “Molecular Probes Handbook of Fluorescent Probes and Research Chemicals” published by Molecular Probes, Inc., Eugene, Oreg. (originally published in 1989).

As used herein, the phrase “detectable response” refers to any change in, or occurrence of, a signal that is detectable, either by observation or instrumentally. In many cases, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

As used herein, the term “monovalent,” in reference to antibody fragments, refers to an antibody fragment that has only one antigen-binding site. Examples of monovalent antibody fragments include, but are not limited to, Fab fragments (no hinge region), Fab′ fragments (monovalent fragments that contain a heavy chain hinge region), and single-chain fragment variable (ScFv) proteins.

As used herein, “target” refers to any substance to be detected by its association with a target-binding antibody.

Other terms are defined in other portions of this description, even though not included in this subsection.

### Detection Methods

In at least one aspect, the disclosure provides methods for determining the presence of a target compound in a sample, the methods comprising: providing (a) a target-binding antibody comprising a region that binds non-covalently to a target compound, (b) a quenching composition comprising quenching compounds, wherein the quenching compounds comprise serum components, and (c) a labeling composition comprising labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds; introducing the target-binding antibody into the labeling composition to form an active product composition comprising (a) a labeled antibody complex, which comprises the target-binding antibody and one or more labeling proteins bound non-covalently to the target-binding antibody, and (b) free labeling proteins, which are not bound non-covalently to any target-binding antibodies; introducing the quenching composition to the active product composition to form a quenched active product composition comprising the labeled antibody complex and quenched labeling proteins, wherein each of the quenched labeling proteins comprises one or more quenching compounds bound non-covalently to one or more labeling proteins; and introducing at least a portion of the quenched active product composition to a sample; and following the introduction of at least a portion of the quenched active product composition to the sample, analyzing the sample to determine the presence or absence of the target compound in the sample.

As noted above, the disclosure provides methods that use a target-binding antibody comprising a region that binds non-covalently to a target compound. As used herein, the term “binds” refers to a non-covalent association having a binding constant, Kb, of at least 1×10² M⁻¹, but often higher, such as at least 1×10³ M⁻¹, or at least 1×10⁴ M⁻¹, or at least 1×10⁵ M⁻¹, or at least 1×10⁶ M⁻¹. The target-binding antibody can be any suitable antibody, as described above, so long as it is capable of binding with the target compound. In general, the target-binding antibody selectively binds in a non-covalent manner with the target compound, meaning that it binds to the target compound with a greater affinity that to other substances present in the sample. For example, in some such embodiments, the target-binding antibody binds to the target with a binding constant, Kbₜ, at least 100 times greater than the binding constant, Kbₒ, with which the target-binding antibody binds to other substances in the sample. In other examples, Kbₜ is at least 90, at least 80, at least 70, at least 60, at least 50, at least 40, at least 30, at least 20, or at least 10 times greater than Kbₒ. Any suitable region of the target-binding antibody can bind to the target compound. In some embodiments, however, the Fab region of the target-binding antibody comprises the region that binds to the target compound.

The target compound can be any analyte whose presence or concentration one may desire to determine in a sample, provided an antibody can be produced to non-covalently bind with the analyte. Non-limiting examples of a suitable target compound include, but are not limited to, a protein, an antibody, a peptide, an oligopeptide, a glycoprotein, an enzyme, an enzyme substrate, a hormone, a lymphokine, a metabolite, an antigen, a hapten, a lectin, avidin, streptavidin, a toxin, a poison, an environmental pollutant, a carbohydrate, a carbohydrate, an oligosaccharide, a polysaccharide, a lipid, a glycolipid, a nucleotide, an oligonucleotide, a nucleic acid, a derivatized nucleic acid (such as a deoxyribo- or ribonucleic acid, or a peptide nucleic acid), a DNA fragment, an RNA fragment, a derivatized DNA or RNA fragment (such as a single- or multi-stranded fragment), a natural or synthetic drug, a receptors, a virus particle, a bacterial particle, a virus component, a biological cell, a cellular component (such as a cellular membrane or an organelle), a natural or synthetic lipid vesicle, and a polymer membrane.

As also noted above, the disclosure provides methods that use a quenching composition comprising quenching compounds. The quenching compounds include one or more serum components, such as the components of the serum of an animal, such as a human. In some embodiments, the serum components are human serum components. In some embodiments, the quenching composition comprises the serum components (for example, alpha globulins, beta globulins, gamma globulins, or some combination thereof) at a higher concentration than their natural (i.e., native) concentration in serum. In some such embodiments, the serum components (for example, alpha globulins, beta globulins, gamma globulins, or some combination thereof) are present in the quenching composition at a concentration that is at least 1.5 times higher, or at least 2.0 times higher, or at least 3.0 times higher, or at least 4.0 times higher, than their native, unaltered concentration in serum.

In some embodiments, the identities and relative concentrations of the serum components in the quenching composition are similar to that of serum. In other embodiments, they are different. For example, in some embodiments, the quenching composition comprises fractionated serum components, which are the serum components that remain following fractionation of serum with a fractionating fractionating agent or method. Any fractionating agent or fractionating method known in the art may be used. Some non-limiting examples of fractionating agents include kosmotropic salts (e.g., ammonium sulfate) or selective precipitants (e.g. caprylic acid). Some non-limiting examples of fractionating methods include fractionating chromatography methods, such as affinity chromatography (e.g., Protein A chromatography), ion exchange chromatography (e.g. DEAE chromatography), hydrophobic interaction chromatography (e.g. phenyl chromatography) or multimodal chromatography (e.g. hydroxyapatite). In certain embodiments, the fractionation removes most or all of certain serum components. For example, in some embodiments, the fractionated serum components have a reduced concentration of albumin. Thus, in some embodiments, the quenching composition comprises albumin at a concentration of no more than 25 mg/mL, or no more than 20 mg/mL, or no more than 15 mg/mL, or no more than 10 mg/mL, or no more than 5 mg/mL, or no more than 2 mg/mL, or no more than 1 mg/mL.

In some embodiments of any of the foregoing embodiments, fractionating does not remove a substantial portion of the alpha globulins, beta globulins, and gamma globulins from serum. For example, in some embodiments, the quenching composition comprises compounds of each naturally occurring subclass of human immunoglobulin G. As noted above, in some such embodiments, these compounds are present at concentrations greater than their native concentration in human serum. In some other embodiments of any of the foregoing embodiments, the quenching composition comprises one or more human immunoglobulins of an isotype other than immunoglobulin G, such as human immunoglobulin A, human immunoglobulin D, human immunoglobulin E, or human immunoglobulin M. In some embodiments, the quenching composition comprises human immunoglobulin A, human immunoglobulin M, or a mixture thereof. In some such embodiments, such immunoglobulins are present in the quenching composition at concentrations similar to their native concentration in human serum, or are present in the quenching composition at concentrations greater than their native concentration in human serum, for example, 1.5 times or more greater, or 2.0 times or more greater, or 3.0 times or more greater, or 4.0 times or more greater.

According to the embodiments set forth above, the quenching composition differs from an animal serum, such as human serum, in the concentration of certain components. For example, the quenching composition may have a higher concentration of certain serum components than serum (for example, human immunoglobulins), a lower concentration of certain serum components (for example, albumin), or a combination of these features. The quenching composition in some examples differs from purified human immunoglobulin G in that, in certain embodiments, it contains serum components besides human immunoglobulin G, such as human immunoglobulin A, human immunoglobulin M, or a mixture thereof.

As noted above, in certain embodiments, the quenching composition is formed via the fractionation of an animal serum, such as human serum, using a fractionating (precipitating) agent. Such fractionated serum components are obtained by carrying out a fractionating procedure. Suitable fractionating procedures include introducing kosmotropic agents, such as ammonium sulfate or another such agent, to serum to induce the precipitation of certain serum components. Suitable fractionating procedures also include the use of various chromatographic methods, such as fractionating chromatographic methods. Various procedures can also be used in combination. The solution containing non-precipitated serum components is discarded and the precipitated serum components are resolubilized at a concentration 4-5-fold higher than in the neat serum for use in the quenching composition. In another fractionation method using chromatography or the like, unwanted serum components (e.g. albumin) are separated from desired components (e.g. immunoglobulins) and the desired components are concentrated using various common concentration methods to 4-5-fold higher than in neat serum for use in the quenching composition.

The methods of the disclosure also use a labeling composition comprising labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds.

The labeling protein contains one or more peptide chains, such as peptide chains associated with the monovalent Fab antibody fragment, but, in some embodiments, may optionally contain other regions that are not made up of peptide chains. The monovalent Fab antibody fragment is an antibody fragment, as defined above, derived from the Fab portion of an antibody. The Fab antibody fragment can be derived from a monoclonal antibody or a polyclonal antibody. In some embodiments, it is derived from a polyclonal antibody. The labeling protein includes a region that binds to the target-binding antibody. In this context, the term “bind” has the same meaning as set forth above. In general, that binding region is comprised by the monovalent Fab antibody fragment. The binding region binds to any suitable portion of the target-binding antibody, including the Fc portion or various epitope-presenting portions, such as an Fab or Fab′ portion. In some such embodiments, the binding region of the labeling protein binds to a portion of the target-binding antibody that is at least partially not within the Fc region. In some such embodiments, the binding region of the labeling protein binds to a portion of the target-binding antibody that is not within the Fc portion, e.g., is within the Fab portion or other epitope-presenting portions of the antibody.

The labeling protein is covalently linked to a label. Any suitable label can be used, according to the description above concerning labels. In some embodiments, the label is a fluorescent dye, a phosphorescent dye, a tandem dye, a particle, a nanoparticle, an electron transfer agent, biotin, a hapten, an enzyme, or a radioisotope. The label may be linked to the labeling protein by any suitable means. In some examples, the label is bonded covalently directly to the Fab antibody fragment. In some other embodiments, there can be an intervening linker, which can be made up of amino acids or other suitable building blocks.

As noted above, the labeling proteins also include a region that is capable of binding non-covalently to one or more of the quenching compounds (according to any of the above embodiments). In general, the binding of the labeling proteins to the quenching compounds occurs with a binding constant, Kb, of at least 1×10¹ M⁻¹, but sometimes higher, such as at least 1×10² M⁻¹, or at least 1×10³ M⁻¹, or at least 1×10⁴ M⁻¹, or at least 1×10⁵ M⁻¹, or at least 1×10⁶ M⁻¹. The labeling proteins may bind to any suitable region of the quenching compounds.

The labeling proteins are included within a labeling composition. This composition can be a liquid or solid, although is generally a liquid. The composition can include a number of other suitable substances, as would be suitable for the maintenance of antibody fragments, such as an aqueous carrier, a buffer, various salts, various surfactants, and the like.

The methods disclosed herein include the step of introducing the target-binding antibody into the labeling composition to form an active product composition comprising (a) a labeled antibody complex, which comprises the target-binding antibody and one or more labeling proteins bound non-covalently to the target-binding antibody, and (b) free labeling proteins, which are not bound non-covalently to any target-binding antibodies.

Upon introduction of the target-binding antibodies (for example, as part of an aqueous medium) to the labeling composition, the labeling proteins bind to target-binding antibodies, so as to create a labeled antibody complex that includes the target-binding antibody bound non-covalently with one or more labeling proteins. In some embodiments, at least a portion of the labeled antibody complexes have two or more labeling proteins bound to the target-binding antibodies. In general, it is desirable to ensure a high degree of labeling, such that the active product composition contains few target-binding antibodies that are not bound non-covalently to at least one labeling protein. For example, in some embodiments, following incubation for a suitable period of time (for example, at least 20 or 30 minutes), no more than 5%, or no more than 2%, or no more than 1% of the target-binding antibodies in the active product composition are not bound to at least one labeling protein. In some embodiments, at least 95% of the target-binding antibodies in the active product composition are bound non-covalently to at least one labeling protein. In other embodiments, at least 96%, at least 97%, at least 98%, at least 99%, or at least 99.5% of the target-binding antibodies in the active product composition are bound non-covalently to at least one labeling protein

In general, the active product composition includes an excess of labeling protein, for example, to ensure effective labeling of the target-binding antibodies. In some embodiments, the molar ratio of labeling proteins to target-binding antibodies in the active product composition is at least 2:1, or at least 3:1, or at least 4:1, or at least 5:1.

Due to the use of an excess of labeling protein, the active product composition generally contains free labeling proteins, which are not bound to any target-binding antibodies. These free labeling proteins may have an affinity to certain endogenous antibodies within the sample, so it is desirable to quench them (i.e., bind them to another protein) prior to the introduction of the composition to the sample. Therefore, the methods disclosed herein include a step of introducing a quenching composition to the active product composition to form a quenched active product composition comprising the labeled antibody complex and quenched labeling proteins, wherein each of the quenched labeling proteins comprises one or more quenching compounds bound non-covalently to one or more labeling proteins.

Quenching compositions are discussed in detail above. To ensure effective quenching of free labeling protein, quenching compounds are generally introduced to the active product composition at an excess relative to the free labeling protein. In some embodiments, the quenching composition is introduced such that there is at least an excess of quenching compounds on a molar basis to free labeling protein in the active product composition. In some such embodiments, the quenching compounds are introduced in amounts of at least a 50-fold excess, or at least a 100-fold excess, or at least a 150-fold excess, or at least a 200-fold excess, or at least a 250-fold excess, or at least a 500-fold excess, or at least a 1000-fold excess, or at least a 2000-fold excess, or at least a 3000-fold excess, or at least a 4000-fold excess, or at least a 5000-fold excess. In some embodiments, following introduction of the quenching composition, less than 1.0%, or less than 0.5%, or less than 0.1% of the labeling protein exists as free labeling protein. The rest is bound either to the target-binding antibody or to quenching compound(s).

In some cases, especially where the sample is a biological sample, it can be desirable to treat the sample with a blocking composition before introducing labeled target-binding antibody to the sample. Thus, in some embodiments, the methods disclosed herein include a step of introducing a blocking composition to the sample prior to the introduction of a composition containing the labeled target-binding antibody. Any suitable blocking composition can be used, and may be selected depending on the nature and composition of the sample. In some embodiments, the blocking composition comprises a non-animal protein, such as a vegetable protein, for example, rice protein. In other embodiments, the blocking composition can include certain animal proteins, such as bovine serum albumin (BSA). In general, any suitable protein-based composition of animal or plant origin can be used, as it helps to block endogenous antibodies that may be present within the sample, and, preferable, does not bind with any substantial affinity to the target compound within the sample.

Following an optional blocking step, the quenched active composition is introduced to the sample, which permits the labeled target-binding antibodies to bind to any target compounds within the sample. The methods disclosed herein are not limited to being practiced with any particular sample. Thus, any suitable sample can be used. In some embodiments, however, the sample is the tissue of an animal, such as the tissue of a mammal (e.g., human). In some embodiments, the target-binding antibody is of the same species as the sample, such as both human, or both mouse, and the like. The tissue can be solid or a fluid. In some embodiments, the sample is a solid, such as tissue from a solid organ or a growth, such as a solid tumor. In other embodiments, the sample is a fluid, such as blood, sputum, cerebral fluid, and the like.

Following introduction of the quenched active product composition to the sample, it can be desirable to wash the sample to remove any labeled compounds that did not bind to a target compound (e.g., following a suitable period of incubation). The wash can be accomplished by any suitable means, depending on the nature of the sample, the concentration of the target compound, and other factors known to those of skill in the art.

Following the introduction of the quenched active product composition to the sample and the optional washing, the methods disclosed herein include analyzing the sample to determine the presence or absence of the target compound in the sample. The analysis will depend on a number of factors, such as the identity of the label, the nature of the sample, etc. This analysis need not occur via a single step. For example, in some instances, the analysis may occur through multiple steps, where the presence of the label is determined indirectly through an effect that is induced by the presence of the label.

Further modifications and augmentations of the methods disclosed herein will be appreciated by the person of ordinary skill in the art, and are implicitly included within the scope of what is disclosed here.

### Systems for Detection

In at least another aspect, the disclosure provides a systems, the systems comprising: (a) a target-binding antibody comprising a region that binds non-covalently to a target compound, (b) a quenching composition comprising quenching compounds, wherein the quenching compounds comprise serum components, and wherein the serum components are at a concentration greater than that normally found in serum and (c) a labeling composition comprising labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds. These aspects include various embodiments, according to those set forth immediately above with reference to the methods, which are hereby incorporated into this section by reference.

In some embodiments, the systems disclosed herein also include a blocking composition, according to any of the embodiments set forth above with respect to the methods.

### Kits for Detection

In at least another aspect, the disclosure provides kits, the kits comprising: (a) a quenching composition comprising quenching compounds, wherein the quenching compounds comprise serum components, and wherein the serum components are at a concentration greater than that normally found in serum, and (c) a labeling composition comprising labeling proteins, wherein each of the labeling proteins comprises a monovalent Fab antibody fragment comprising a region that binds non-covalently to a target-binding antibody, and wherein each of the labeling proteins is linked covalently to one or more labels, and wherein each of the labeling proteins is monovalent and capable of binding non-covalently to at least one of the quenching compounds. These aspects include various embodiments, according to those set forth immediately above with reference to the methods, which are hereby incorporated into this section by reference.

In some embodiments, the kits disclosed herein also include a blocking composition, according to any of the embodiments set forth above with respect to the methods.

### EXAMPLES

### Example 1—Fractionation of Human Serum for Quenching Reagent

Human serum (470 mLs) was adjusted to 50% saturation with solid ammonium sulfate. The resulting precipitate was collected by centrifugation and the supernatant was discarded. The pelleted precipitate was resuspended in approximately 50 mLs of Phosphate-buffered saline (PBS) buffer. The resuspended pellet was dialyzed against PBS buffer to remove remaining ammonium sulfate. Analysis of the optical density (a rough quantitation of the serum protein content) showed a decrease of nearly 75% of protein. SDS-PAGE analysis indicates the majority of the protein removed is serum albumin. Immunoglobulin G and other unidentified serum proteins were enriched in this process by approximately 4-fold. This solution was used as the quenching reagent in Examples 2-5.

### Example 2—Detecting Human Desmin with a Humanized Antibody in Human Frozen Colon

Preparation of Antibody Staining Complex—

Humanized anti-Desmin (primary) antibody was diluted at 1:50 into 250 uL RTU Animal-Free Block and Diluent containing HRP-Fab anti-human IgG and incubated for 30 minutes. After 30 minutes, 250 microliters (μl) ammonium sulfate fractionated serum was added to the primary antibody—HRP-Fab anti-human IgG solution, mixed and incubated for 30 minutes. The final anti-Desmin dilution is 1:100. The solution is now ready to use.

A negative control staining complex was prepared as above except no anti-Desmin (primary) antibody was added.

Preparation of Frozen Human Tissue—

Acetone fixed, 5-micron frozen human colon tissue sections were treated with an endogenous peroxidase blocking solution for 10 minutes. The sections were rinsed with PBS then blocked with a rice protein based blocking solution for 10 minutes. Blocking reagent was tipped off and the section were processed as described for Panel A, B and C.

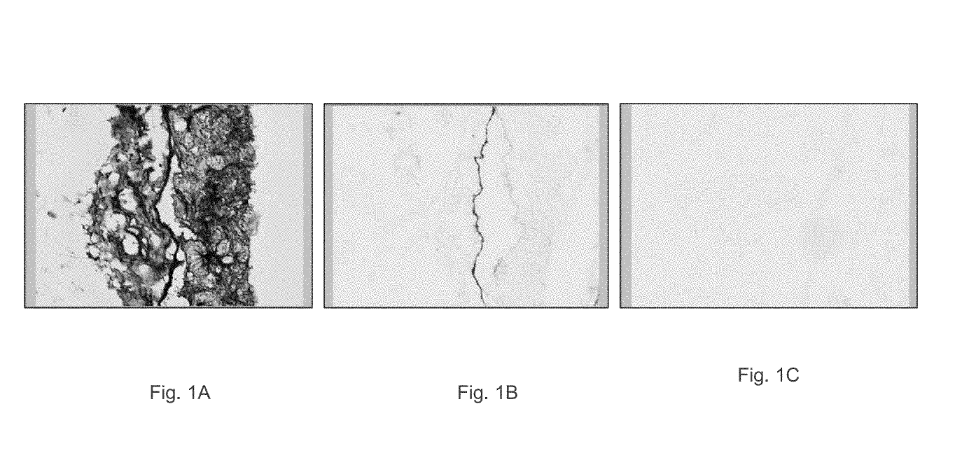

FIG. 1A, Traditional Staining with Primary Antibody:

Human anti-Desmin was applied at a 1:100 dilution in rice protein based blocking solution for 30 minutes. Sections were washed 3 times for 5 minutes each time in PBS. Sections were incubated in HRP-Fab anti-human IgG diluted in rice protein based blocking solution for 30 minutes. Sections were washed in in PBS and stained with DAB as below.

FIG. 1B, Human on Human Kit with Primary Antibody:

Sections were incubated in the antibody staining complex (containing primary antibody) for 30 minutes. Sections were washed in PBS and stained with DAB as below.

FIG. 1C, Human on Human Kit without Primary Antibody:

Sections were incubated in the antibody staining complex (without primary antibody) for 30 minutes. Sections were washed in PBS and stained with DAB as below.

FIGS. 1A-1C DAB Staining and Mounting.

All sections were developed using DAB substrate. Sections were rinsed in water, dehydrated through graded alcohol and xylene and mounted in permanent mounting media.

Using a traditional staining method, the specific desmin location cannot be identified because the background staining is too dark, as shown in FIG. 1A. Using the antibody complex method, the background staining is removed allowing specific signal to be identified, as shown in FIG. 1B. Specific signal is present only when the primary antibody is used; no primary antibody was used as a control, as shown in FIG. 1C.

### Example 3—the New Technology Improves Signal to Noise Over Existing Technology

### Preparation of Antibody Staining Complex—New Technology

Humanized anti-Desmin (primary) antibody was diluted at 1:50 into 250 μl RTU Animal-Free Block and Diluent containing HRP-Fab anti-human IgG and incubated for 30 minutes. After 30 minutes, 250 μl ammonium sulfate fractionated serum was added to the primary antibody—HRP-Fab anti-human IgG solution, mixed and incubated for 30 minutes. Final primary dilution is 1:100. The solution is now ready to use.

### Preparation of Antibody Staining Complex—Existing Technology

Humanized anti-Desmin (primary) antibody was diluted at 1:100 into RTU Human Primer overnight at 4° C. On the next day, this solution added the Quenching Buffer to the primary antibody solution and incubated 30 minutes. The solution is now ready to use.

Preparation of FFPE Human Tissue—

5-micron, antigen-retrieved human colon tissue sections were treated with an endogenous peroxidase blocking solution for 10 minutes. The sections were rinsed with PBS then further processed as indicated in Panels A, B and C.

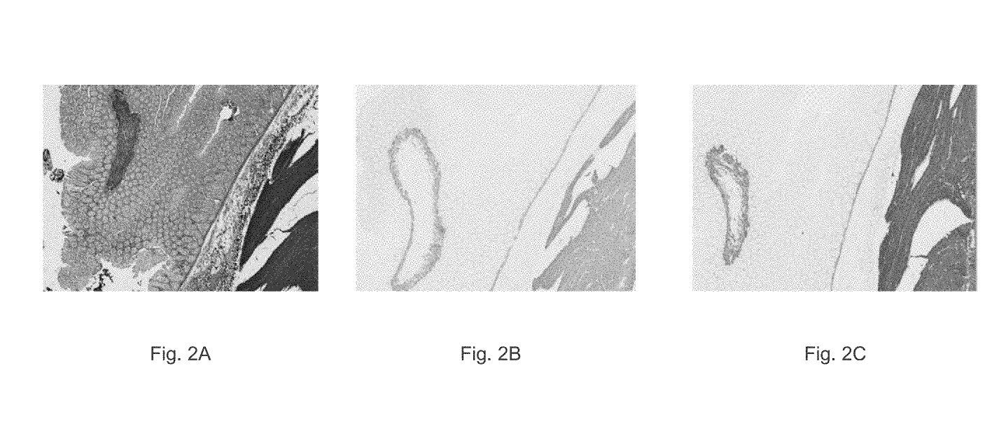

FIG. 2A.

The sections were blocked with a rice protein based blocking solution for 10 minutes. Blocking reagent was tipped off and then Human anti-Desmin was applied at a 1:100 dilution in rice protein based blocking solution for 30 minutes. Section were washed 3×5 minutes in PBS. Sections were incubated in HRP-Fab anti-human IgG diluted in rice protein based blocking solution for 30 minutes. Sections were washed in in PBS and stained with DAB as below.

FIG. 2B.

The sections were blocked with Block A for 20 minutes then rinsed with PBS. The sections were incubated in Block B for 5 minutes then rinsed in PBS. Sections were incubated in the antibody staining complex from the Existing Technology (with primary antibody) for 30 minutes. Sections were rinsed with PBS then incubated in HRP Polymer antibody for 10 minutes. Sections were washed in PBS and stained with DAB as below.

FIG. 2C.

Sections were incubated in the antibody staining complex from the New Technology (containing primary antibody) for 30 minutes. Sections were washed in PBS and stained with DAB as below.

DAB Staining and Mounting.

All sections were developed using DAB substrate. Sections were rinsed in water, dehydrated through graded alcohol and mounted in permanent mounting media.

Using a traditional staining method, the specific desmin location cannot be identified because the background staining is too dark, as show in FIG. 2A. Using the antibody complex from the New Technology, the background staining is removed allowing specific signal to be identified, as shown in FIG. 2B. The specific signal in FIG. 2B is 2-4 times darker than the specific signal seen with the Existing Technology, shown in FIG. 2C.

### Example 4—the New Technology Improves Signal to Noise Over Comparative Examples

The inventive technology was prepared as Inventive Sample 1, and compared to conventional staining kits, which are Comparative Samples 2-4. The preparation of an antibody staining complex for each example is detailed below. All incubations were conducted at room temperature (approximately 25° C.) unless otherwise noted. The primary antibody was Humanized anti-Desmin antibody, which is commercially available as BioRad HCA023, Lot 1605.

Preparation of FFPE Human Tissue—

5-micron, antigen-retrieved human colon tissue sections were treated with an endogenous peroxidase blocking solution for 10 minutes. The sections were rinsed with PBS then further processed as indicated below in the Staining Procedures.

Staining and Mounting.

All sections were stained as detailed below. Sections were rinsed in water, dehydrated through graded alcohol and mounted in permanent mounting media.

### Inventive Sample 1

Preparation of Primary Antibody Staining Complex—

Humanized anti-Desmin (primary) antibody (Reagent A) was diluted into 250 μl RTU Animal-Free Block and Diluent containing HRP-Fab anti-human IgG at 1:50 (10 μl into 250 μl), 1:150 (3.3 μl into 250 μl), and 1:450 (1.1 μl into 250 μl) dilutions and incubated for 30 minutes. After 30 minutes, 250 μl ammonium sulfate fractionated serum (Reagent B) was added to dilution, mixed and incubated for 30 minutes. Reagents A and B were prepared as in Examples 1 and 2, above. The solutions are now ready to use. Total elapsed time for preparing the primary antibody staining complex was 60 minutes.

Staining Procedure—

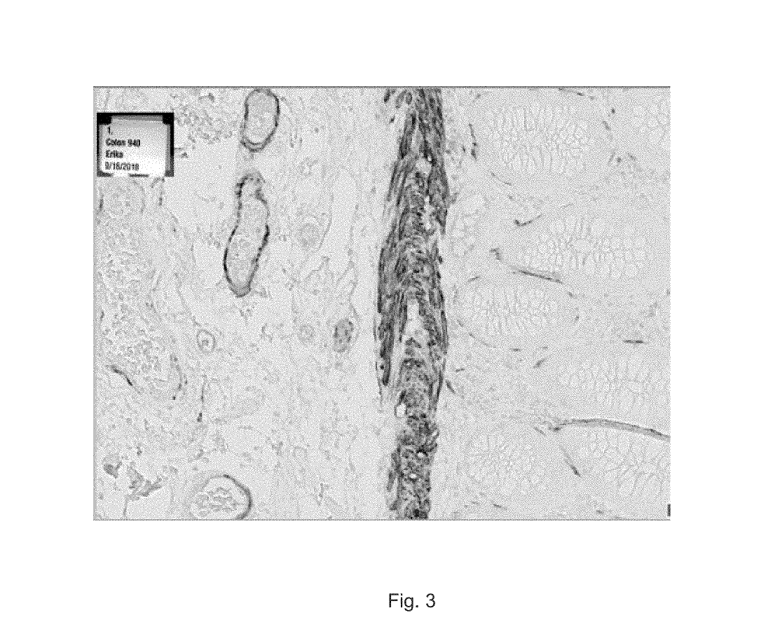

5-micron, antigen-retrieved human colon tissue sections were treated with Bloxall™ endogenous peroxidase blocking solution (available from Vector Labs, Burlingame, Calif.) for 10 minutes. The sections were rinsed with PBS, then treated with RTU Animal Free Blocking Agent. The sections were incubated for 15 minutes, and then tipped off block. The primary antibody staining complex of Sample 1 was added, followed by incubation for 30 minutes. The sections were washed with fresh PBS for 5 minutes, and then ImmPACT™ DAB EqV working solution (available from Vector Labs, Burlingame, Calif.) was added, followed by incubation for 8 minutes. The sections were rinsed in tap water and then imaged. Total elapsed time for the staining procedure was 1 hour 8 minutes. Results of staining with the 1/150 dilution are shown in FIG. 3. All dilutions provided acceptable results and thus the methods are applicable for a range of primary antibody concentrations.

Any solutions not provided in the commercially available kits were provided by Vector Labs, Burlingame, Calif.

### Comparative Sample 2

A kit commercially available as Human-on-Human HRP-Polymer from Biocare Medical, Pacheo, Calif. was acquired.

Preparation of Antibody Staining Complex—

Humanized anti-Desmin (primary) antibody was diluted into 250 μl RTU Animal-Free Block and Diluent containing HRP-Fab anti-human IgG at 1:50 (10 μl into 200 μl), 1:150 (3.3 μl into 200 μl), and 1:450 (1.1 μl into 200 μl) dilutions; 40 μl Dig anti-Human Linker was added to each dilution and incubated for 60 minutes. Next, 40 μl Human Adsorption reagent was added to each dilution, mixed, and the resulting solutions were vortexed for 5 seconds and then incubated for 30 minutes. Each dilution was additionally diluted to a final volume of 500 μl with RTU Animal Free Block and Diluent. The solutions are now ready to use. Total elapsed time for preparing the primary antibody staining complex was 90 minutes.

Staining Procedure—

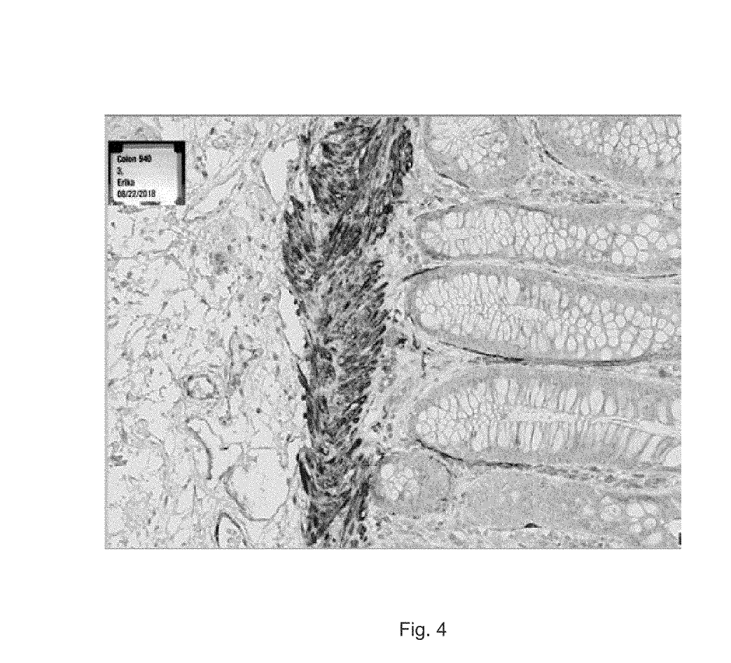

5-micron, antigen-retrieved human colon tissue sections were treated with Bloxall endogenous peroxidase blocking solution for 10 minutes. The sections were rinsed with PBS for 1 minute, then treated with the primary antibody staining complex of Sample 2. The sections were incubated for 15 minutes, and then treated with Hum Ab complex. After incubating for 60 minutes, the sections were washed with fresh PBS for 5 minutes, and then Mouse anti-Dig was added, followed by a 15 minute incubation. The sections were washed with fresh PBS for 5 minutes, and then RTU Mach 2 HRP anti-Mouse was added, followed by a 30 minute incubation. The sections were washed with fresh PBS for 5 minutes, and then ImmPACT DAB Equivalent working solution was added, followed by incubation for 8 minutes. The sections were rinsed in tap water and then imaged. Total elapsed time for the staining procedure was 1 hour 33 minutes. Results of staining with the 1/150 dilution are shown in FIG. 4.

### Comparative Sample 3

A kit commercially available as Human on Human IHC Kit (HRP/DEB) from Abcam, Cambridge, UK was acquired.

Preparation of Antibody Staining Complex—

Humanized anti-Desmin (primary) antibody was diluted into Reagent 1 at 1:50 (10 μl into 500 μl), 1:150 (3.3 μl into 500 μl), and 1:450 (1.1 μl into 500 μl) dilutions and incubated overnight at 4° C., then allowed to warm to room temperature, then 100 μL Reagent 2 was added to each dilution. The solutions are now ready to use. Total elapsed time for preparing the primary antibody staining complex was overnight.

Staining Procedure—

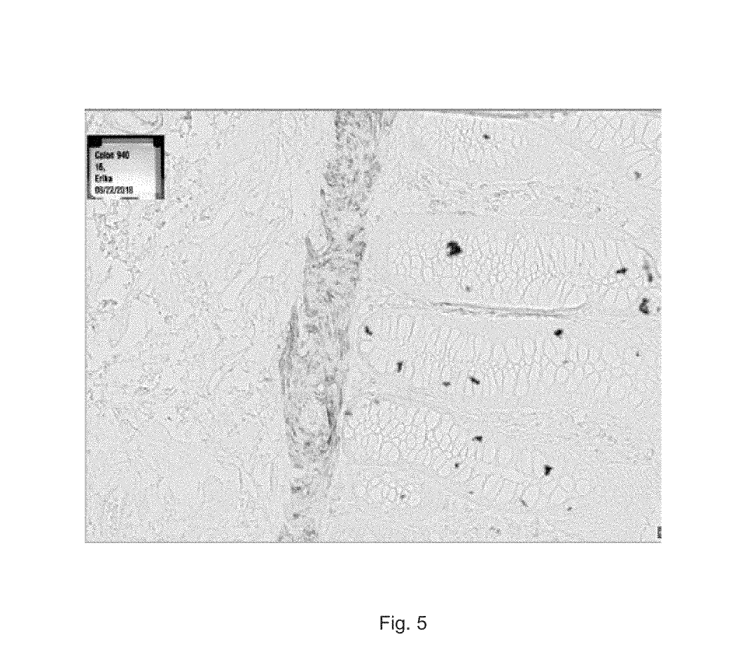

5-micron, antigen-retrieved human colon tissue sections were treated with Bloxall endogenous peroxidase blocking solution for 10 minutes. The sections were rinsed with Phosphate Buffered Saline with Tween 20 buffer (PBST) then treated with Block A. The sections were incubated for 30 minutes, and washed with PBST three times for two minutes each time. The sections were then treated with Block B, incubated for 5 minutes, and washed with PBST three times for two minutes each time. The sections were then treated with the antibody staining complex of Sample 3. After incubating for 60 minutes, the sections were washed with PBST three times for two minutes each time, and then RTU Human HRP Polymer was added, followed by a 10 minute incubation. The sections were washed with PBST three times for two minutes each time, and then DAB solution was added, followed by incubation for 5 minutes. The sections were rinsed in tap water and then imaged. Total elapsed time for the staining procedure was 1 hour 24 minutes. Results of staining with the 1/150 dilution are shown in FIG. 5.

### Comparative Example 4

A kit commercially available as Human-to-Human Blocking Reagent from Scytek, West Logan, Utah was acquired.

Preparation of Antibody Staining Complex—

Humanized anti-Desmin (primary) antibody was diluted into Animal-Free Block and Diluent at 1:50 (10 μl into 50 μl), 1:150 (6.6 μl into 500 μl), and 1:450 (1.1 μl into 500 μl) dilutions. The solutions are now ready to use.

Staining Procedure—

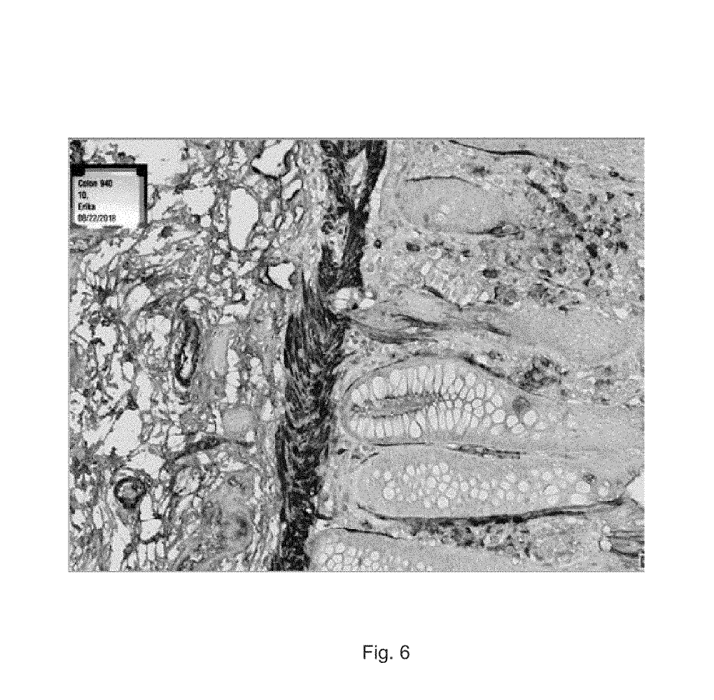

5-micron, antigen-retrieved human colon tissue sections were treated with Bloxall endogenous peroxidase blocking solution for 10 minutes. The sections were rinsed with PBS, then treated with RTU Animal Free Blocking Agent. The sections were incubated for 15 minutes, and then tipped off block. The sections were treated with Human-to-Human Block and incubated for 60 minutes, then rinsed four times with PBS. The sections were then treated with the antibody staining complex of Sample 4. After incubating for 30 minutes, the sections were washed with fresh PBS four times, and then polymer anti-Human IgG was added, followed by a 30 minute incubation. The sections were washed with fresh PBS four times, and then ImmPACT DAB Equivalent working solution was added, followed by incubation for 8 minutes. The sections were rinsed in tap water and then imaged. Total elapsed time for the staining procedure was 1 hour 8 minutes. Results of staining with the 1/150 dilution are shown in FIG. 6.

Compared to Comparative Samples 2-4, Inventive Sample 1 procedure required fewer staining procedure steps, could be conducted in less time, and provided images with excellent signal-to-noise ratios.

### Example 5—the New Technology has Acceptable Negative Staining

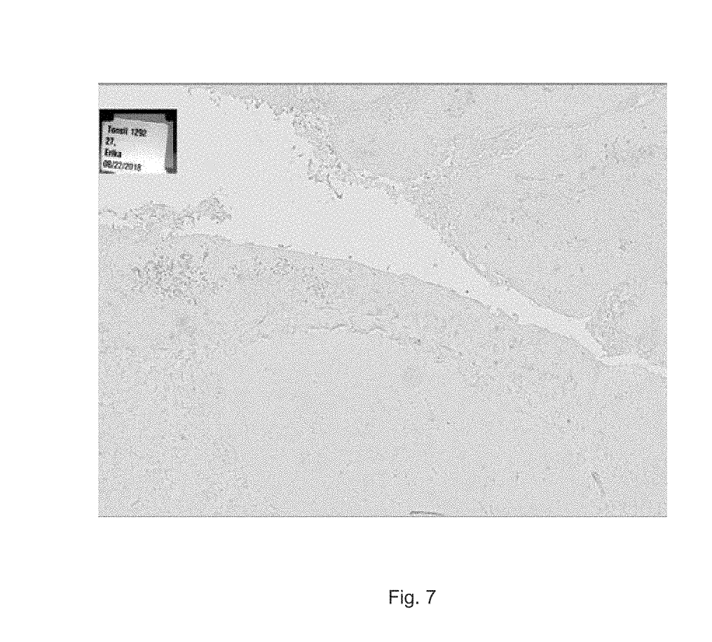

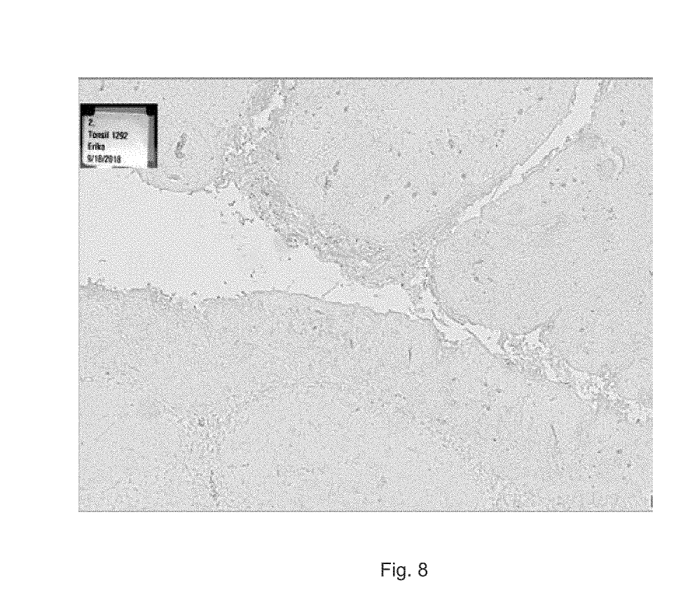

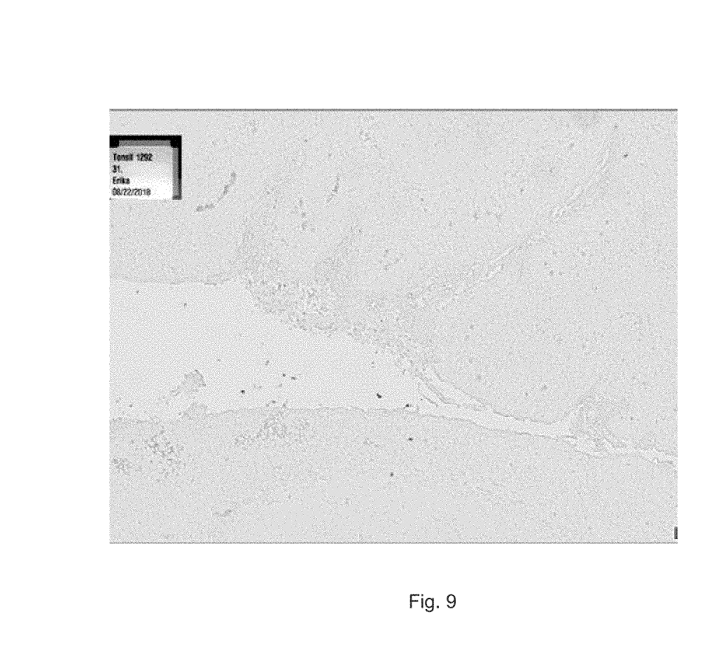

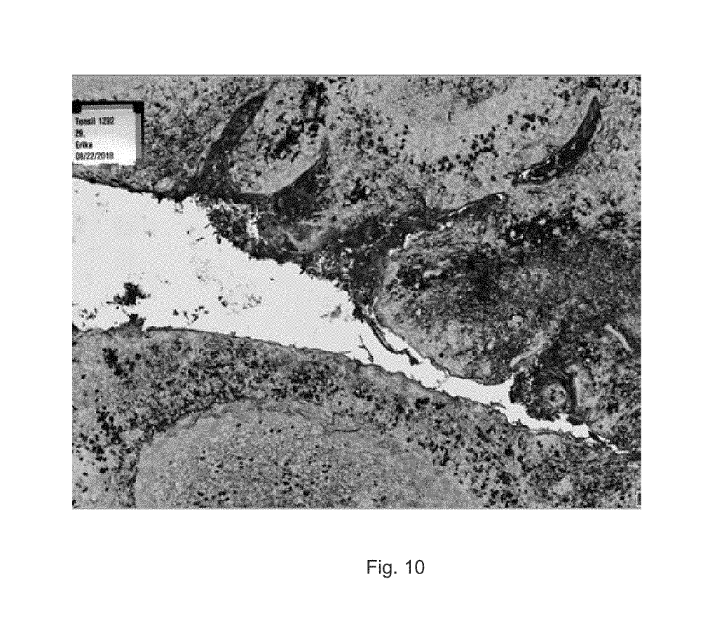

Inventive Sample 1 and Comparative Samples 2-4 were prepared as in Example 4, except that the tissue sections were Human FFPE Tonsil, and no primary antibody staining complex was added. There should be no dark staining in the images of these samples, as dark staining indicates a false positive result. The section images for Sample 1 is shown in FIG. 7, and for Comparative Samples 2-4 are shown in FIGS. 8-10, respectively. In particular, Comparative Sample 4 showed high negative staining, while Inventive Sample 1 and Comparative Samples 2 and 3 shown acceptable staining.

1. A method for determining the presence of a target compound in a sample, the method comprising:
providing (a) a target-binding antibody comprising a region that binds non-covalently to a target compound, (b) a quenching composition comprising quenching compounds, wherein the quenching compounds comprise enriched serum components, and wherein the enriched serum components are at a concentration greater than 4-fold that normally found in serum and (c) a labeling composition comprising one or more labeling proteins, wherein each of the labeling proteins comprises a monovalent Fab antibody fragment comprising a region that binds non-covalently to the target-binding antibody, and wherein each of the labeling proteins is linked covalently to one or more labels, and wherein each of the labeling proteins is monovalent and capable of binding non-covalently to at least one of the quenching compounds;
introducing the target-binding antibody into the labeling composition to form an active product composition comprising (a) a labeled antibody complex, which comprises the target-binding antibody and one or more labeling proteins bound non-covalently to the target-binding antibody, and (b) free labeling proteins, which are not bound non-covalently to any target-binding antibodies;
introducing the quenching composition to the active product composition to form a quenched active product composition comprising the labeled antibody complex and quenched labeling proteins, wherein each of the quenched labeling proteins comprises one or more quenching compounds bound non-covalently to one or more labeling proteins; and
introducing at least a portion of the quenched active product composition to a sample; and
following the introduction of at least a portion of the quenched active product composition to the sample, analyzing the sample to determine the presence or absence of the target compound in the sample.

2. The method of claim 1, wherein the target compound is selected from the group consisting of a protein, an antibody, a peptide, an oligopeptide, a glycoprotein, an enzyme, an enzyme substrate, a hormone, a lymphokine, a metabolite, an antigen, a hapten, a lectin, avidin, streptavidin, a toxin, a poison, an environmental pollutant, a carbohydrate, a carbohydrate, an oligosaccharide, a polysaccharide, a lipid, a glycolipid, a nucleotide, an oligonucleotide, a nucleic acid, a derivatized nucleic acid, a DNA fragment, an RNA fragment, a derivatized DNA or RNA fragment, a natural or synthetic drug, a receptors, a virus particle, a bacterial particle, a virus component, a biological cell, a cellular, a natural or synthetic lipid vesicle, and a polymer membrane.

3. The method of claim 1, wherein the sample is a tissue of a mammal.

4. The method of claim 3, wherein the target binding antibody is of the same species as the tissue of the mammal.

5. The method of claim 1, wherein the sample comprises a solid tissue.

6. The method of claim 1, wherein the tissue is a fluid comprising blood, sputum, or cerebral fluid.

7. The method of claim 1, wherein the monovalent Fab antibody fragment is derived from a monoclonal antibody or a polyclonal antibody.

8. The method of claim 1, wherein the quenching composition comprises albumin at a concentration of no more than 10 mg/mL.

9. The method of claim 1, wherein the quenching composition comprises one or more naturally occurring subclasses of immunoglobulin G.

10. The method of claim 1, wherein the quenching compounds comprise one or more gamma-globulins, which comprise one or more immunoglobulins of an isotype other than immunoglobulin G.

11. The method of claim 10, wherein the one or more gamma-globulins comprise one or more immunoglobulins selected from the group consisting of immunoglobulin A and immunoglobulin M.

12. The method of claim 1, comprising, before introduction of at least a portion of the quenched active product composition to the sample, introducing a blocking composition to the sample.

13. The method of claim 12, wherein the blocking composition is substantially free of mammalian antibodies and mammalian antibody fragments.

14. The method of claim 12, wherein the blocking composition comprises a non-animal blocking agent.

15. The method of claim 14, wherein the blocking composition comprises a plant protein.

16. The method of claim 15, wherein the plant protein is a rice protein.

17. The method of claim 1, where the quenching composition is formed by a process comprising:
providing a starting composition comprising serum of the same species as the tissue;
fractionating the starting composition to form an enriched composition comprising fractionated serum proteins comprising alpha globulins, beta globulins, and gamma globulins; and
concentrating the fractionated serum proteins to form the quenching composition,
wherein the quenched active product composition comprises a 5000-fold molar excess of the fractionated serum proteins relative to the labeling protein.

18. The method of claim 17, wherein the fractionating step comprises one or more of precipitation with kosmotropic agents, selective precipitants, or ion exchange chromatography.

19. The method of claim 1, wherein a portion of the monovalent Fab antibody fragment binds non-covalently to at least a portion of the target-binding antibody not within an Fc region of the target-binding antibody.

20. The method of claim 1, wherein the label is selected from the group consisting of a fluorescent dye, a phosphorescent dye, a tandem dye, a particle, a nanoparticle, an electron transfer agent, biotin, a hapten, an enzyme, and a radioisotope.
